(19) United States
(12) Patent Application Publication
(10) Pub. No.: US 2023/0288325 A1
(43) Pub. Date: Sep. 14, 2023

(54) METHOD, DEVICE AND COMPUTER READABLE MEDIUM FOR ANOMALY DETECTION OF A SUBSTANCE

(71) Applicant: THE HONG KONG POLYTECHNIC UNIVERSITY, Hong Kong (CN)

(72) Inventors: Lok Ting LAU, Hong Kong (CN); Jian Nong CAO, Hong Kong (CN); Lei ZHANG, Hong Kong (CN); Nelly Yan Yan LAM, Hong Kong (CN); Wengen LI, Shanghai (CN); Billy HE, Hong Kong (CN); Bernard Huan CHANG, Hong Kong (CN); Yee San Issan TAM, Hong Kong (CN); Teresa CHUNG, Hong Kong (CN)

(21) Appl. No.: 17/654,603

(22) Filed: Mar. 14, 2022

(51) Int. Cl. G01N 21/3577; G01N 33/04

(52) U.S. Cl. CPC G01N 21/3577; G01N 33/04; G01N 2021/3595

(57) ABSTRACT

In anomaly detection of a substance, a first set of chemical fingerprints is obtained. Each fingerprint in the first set is indicative of a plurality of physiochemical properties for a normal sample of the substance. The first set is converted into a cluster of data points in a multi-dimensional principal component analysis (PCA) plot. Each dimension of the plot is based on a principal component (PC) corresponding to one of the physiochemical properties. A profile pattern of the cluster of data points is constructed as a prediction model to identify a new sample with a chemical fingerprint outside the profile pattern as an anomaly. The prediction model is optimized using a second set of chemical fingerprints. Each chemical fingerprint in the second set is indicative of the plurality of physiochemical properties for testing samples that include normal testing samples and abnormal ones of the substance.

### TECHNICAL FIELD

The present specification relates broadly, but not exclusively, to methods, devices, and computer readable media for anomaly detection of a substance.

### BACKGROUND

Food adulteration has been a major risk to public health. Some common foods subjected to food adulteration include olive oil, milk, honey, saffron, orange juice, coffee, apple juice, grape wine, vanilla extract, and maple syrup. Take milk for example, the outbreak of infant formulae tainted with melamine in 2008 demonstrated how severe the human tolls were owing to food adulteration. By relying on protein specifications, the fraudsters adulterated the milk protein with nitrogen rich compounds to make the protein values appeared authentic. Other major incidents include the Horsemeat Scandal in the UK, Ireland and Europe in 2013 where food as advertised as containing beef were found to contain undeclared horse meat, and the selling of counterfeit olive oil in Italy in 2009. Hence, an effective protocol to detect previously un-encountered adulterants is desired.

Despite global collaboration, current detection methods are virtually target-oriented as regulated by respective local legal authorities. For example, national standards for raw milk from cow are defined in GB 19301-2010 in China. With all analyses looking for specific chemical substances and concentrations, newly engineered and previously unknown adulterants can evade existing target-oriented (or targeted, as interchangeably used in the present application) testing methods, posing serious threats. Nevertheless, it is impossible to test a product with all available test methods, not to mention that current quality evaluation methods used in food industries are often expensive, requiring specialized infrastructure, and are labour-intensive.

As such, an effective non target-oriented (or non-targeted, as interchangeably used in the present application) protocol to detect previously un-encountered adulterants (that is, anomaly in a substance) is desired.

### SUMMARY

According to an aspect, there is provided a method for anomaly detection of a substance. The method comprises: obtaining a first set of chemical fingerprints, wherein each chemical fingerprint of the first set of chemical fingerprints is indicative of a plurality of physiochemical properties for each sample in a set of normal samples of the substance; converting the first set of chemical fingerprints into a cluster of data points in a multi-dimensional principal component analysis (PCA) plot, wherein each dimension of the multi-dimensional PCA plot is based on a principal component (PC), each PC corresponding to one of the plurality of physiochemical properties; constructing a profile pattern of the cluster of data points as a prediction model configured to identify a new sample with a chemical fingerprint falling outside of the profile pattern as an anomaly; and optimizing the prediction model using a second set of chemical fingerprints, wherein the second set of chemical fingerprints are indicative of the plurality of physiochemical properties for a set of testing samples that include both a plurality of normal testing samples and a plurality of abnormal testing samples of the substance.

According to another aspect, there is provided a device for anomaly detection of a substance. The device comprises: at least one processor; and a memory including computer program code for execution by the at least one processor, the computer program code instructs the at least one processor to: obtain a first set of chemical fingerprints, wherein each chemical fingerprint of the first set of chemical fingerprints is indicative of a plurality of physiochemical properties for each sample in a set of normal samples of the substance; convert the first set of chemical fingerprints into a cluster of data points in a multi-dimensional principal component analysis (PCA) plot, wherein each dimension of the multi-dimensional PCA plot is based on a principal component (PC), each PC corresponding to one of the plurality of physiochemical properties; construct a profile pattern of the cluster of data points as a prediction model configured to identify a new sample with a chemical fingerprint falling outside of the profile pattern as an anomaly; and optimize the prediction model using a second set of chemical fingerprints, wherein the second set of chemical fingerprints are indicative of the plurality of physiochemical properties for a set of testing samples that include both a plurality of normal testing samples and a plurality of abnormal testing samples of the substance.

### BRIEF DESCRIPTION OF THE DRAWINGS

Embodiments and implementations are provided by way of example only, and will be better understood and readily apparent to one of ordinary skill in the art from the following written description, read in conjunction with the drawings.

FIG. 1 is a schematic diagram of a device 100 for anomaly detection of a substance, according to an embodiment.

FIG. 2 is a flow chart illustrating a method 200 for anomaly detection of a substance, according to an embodiment.

FIG. 3A is a principal component analysis (PCA) plot 300 of a cluster of data points. The cluster of data points are converted from a first set of chemical fingerprints for a set of normal samples of the substance.

FIG. 3B is a histogram showing the cluster of data points being ranked into a predetermined number of intervals. based on their respective values of calculated square foot of sum of all squared principal component (PC)s

FIG. 3C shows a profile pattern 350 of the cluster of data points. The profile pattern 350 shows “core” and “skin” of the cluster of data points.

FIG. 4A and FIG. 4B are alternative angles of view showing new data points in the PCA plot 300. The new data points are converted from a second set of chemical fingerprints for a set of testing samples that include both a plurality of normal testing samples and a plurality of abnormal testing samples of the substance.

In particular, FIG. 4A shows that the profile pattern (depicted in dots that form an envelope shape 402) wraps around normal testing samples (depicted in dots 404 that are wrapped by the envelop shape 402) from the testing samples, while FIG. 4B shows that samples (depicted in dots such as dot 406) found outside the profile pattern are abnormal testing samples (FIG. 2B).

FIG. 4C shows a scree plot of cumulative % variances for the profile pattern with various numbers (from 1 to 8 in this example) of principal components (PCs).

FIG. 5 shows a diagram 500 depicting a distribution of squared Mahalanobis distance (MD) scores for each sample in the set of testing samples set. The line 502 represents a threshold squared MD score of 5.4 to distinguish whether a sample falls inside (normal) or outside (abnormal) of the profile pattern.

FIG. 6A is a diagram showing an overall accuracy when iterating a range of squared MD scores for determining a threshold squared MD score.

FIG. 6B is a diagram showing a sensitivity value when iterating a range of squared MD scores for determining a threshold squared MD score.

FIG. 7A is a diagram showing spectral data of raw milk retrieved from Fourier transform infrared (FTIR) spectroscopy. In this diagram, the spectral data is plotted in terms of absorbance vs. wavenumber. In FIG. 7A, arrowed numbers 1 to 8 refer to eight (8) spectrum regions where absorbance values are extracted for hierarchical cluster learning to define true normal samples.

FIG. 7B is a dendrogram showing samples clusters within main branches in hierarchical cluster learning.

FIG. 7C is an un-rooted tree showing that two major clusters are observed by the hierarchical cluster learning. Among the two major clusters, one cluster includes branches #1-3 and the other cluster includes branches #4-7.

FIG. 8 is a heat-map visualizing hierarchical cluster learning of 113 abnormal samples (i.e. anomalies) classified as within national standards and 198 true normal samples. In FIG. 8, numbers on the left of the figure indicate branch numbers of dendrogram for samples, whereas numbers on the bottom of the figure indicate regions from which absorbance values are extracted.

FIG. 9 shows a distribution of different anomaly compositions among the seven branches (TN: True normal, AD: adulterant, N: Nitrogen, Carb: carbohydrate).

FIG. 10A is a diagram showing ratio of true normal samples within respective branches. FIG. 10B is a diagram showing ratio of nitrogen-rich abnormal samples within respective branches. FIG. 10C is a diagram showing ratio of carbohydrate-based abnormal samples within respective branches. FIG. 10C is a diagram showing ratio of buffering reagents as abnormal samples within respective branches. In FIGS. 10A to 10D, dotted line and the accompanying % indicate overall % of true normal (198/311 = 63.7%) in FIG. 10A, nitrogen-rich abnormal samples (26/311 = 8.4%) in FIG. 10B, carbohydrate-based abnormal samples (65/311 = 20.9%) in FIG. 10C, and buffering reagents (22/311 = 7.1%) in FIG. 10D.

FIG. 11A shows a boosting tree of a XGBoost model learned by the present prediction model, according to an embodiment.

FIG. 11B is a diagram showing relative importance of each compositional feature (that is, physiochemical property) f1 - f9 according to the learnt XGBoost model. f1 - f9 stands for fat, protein, total solid, non-fat solid, lactose, relative density, freezing point, and acidity, respectively.

FIG. 12 shows a block diagram of a computer system 1200 suitable for use as a device for anomaly detection of a substance.

FIG. 13 is a diagram showing an embodiment of data flow framework 1300 for converting chemical fingerprints and biological fingerprints into a standardized format. The standardized format includes schemas about numerical and categorical testing results (num_cat_testing), spectral testing results (spec_testing), numerical and categorical testing templates (num_cat-testing_template), and spectral testing results templates (spec_testing_template). It is appreciable to those skilled in the art that the standardized format may include other schemas about other physiochemical properties of samples.

FIG. 14 is a diagram showing an embodiment of the schemas depicted in FIG. 13. In this embodiments, exemplary tables are shown with columns of data captured in the respective schemas num_cat_testing, spec_testing, num_cat-testing_template, and spec_testing_template.

Skilled artisans will appreciate that elements in the figures are illustrated for simplicity and clarity and have not necessarily been depicted to scale. For example, the dimensions of some of the elements in the illustrations, block diagrams or flowcharts may be exaggerated in respect to other elements to help to improve understanding of the present embodiments.

### DETAILED DESCRIPTION

Embodiments will be described, by way of example only, with reference to the drawings. Like reference numerals and characters in the drawings refer to like elements or equivalents.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “obtaining”, “converting”, “constructing”, “optimizing”, “calculating”, “ranking”, “determining”, “ranking”, “iterating”, “training”, or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a computer suitable for executing the various methods / processes described herein will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the specification contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM mobile telephone system. The computer program when loaded and executed on such a computer effectively results in an apparatus that implements the steps of the preferred method.

This specification uses the term “configured to” in connection with systems, devices, and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions. For special-purpose logic circuitry to be configured to perform particular operations or actions means that the circuitry has electronic logic that performs the operations or actions.

Embodiments of the present application provide approaches for non-targeted detection of anomaly in a substance. The approaches utilise machine learning methods to learn chemical fingerprints of a substance so as to construct and optimise a prediction model to identify an anomaly, either previously encountered or un-encountered. In the present application, an anomaly is interchangeably referred to as an abnormal sample or an adulterant (AD).

Such a prediction model provided by the present application provides manifold advantages. Firstly, no additional labour-intensive sample pre-treatment or testing using expensive, specialized infrastructure and machineries are required, which reduces chances of human errors and the associated time-cost. Secondly, no a priori knowledge of presence of an anomaly is needed after training stage of the prediction model, as the chemical fingerprints of longitudinal testing data facilitate to spot changes in milk composition and recognise an anomalous pattern over time. Thirdly, no manual establishment of tolerance levels for known authentic reference products (i.e. true normal samples) is required, as an effective mathematical equation is provided in the present application for establishment of threshold scores (interchangeably referred to as cutoff scores) for defining anomalies, which in turn saves time and improves accuracy for the anomaly detection. Lastly and most importantly, the prediction model is able to recognise the presence of more than one anomaly per sample without any prior knowledge of its composition. Multiple unknown anomalies can co-exist in one sample in reality and interactions between them may result in signal interferences and a completely different chemical fingerprint. A newly encountered chemical fingerprint recognised by the prediction model can be learnt for further characterization, which can address practical problems of food adulteration in real-world scenarios.

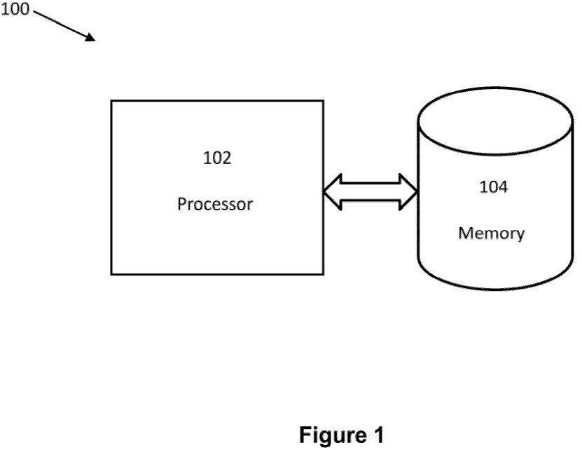

FIG. 1 illustrates a schematic diagram of a device 100 for anomaly detection of a substance.

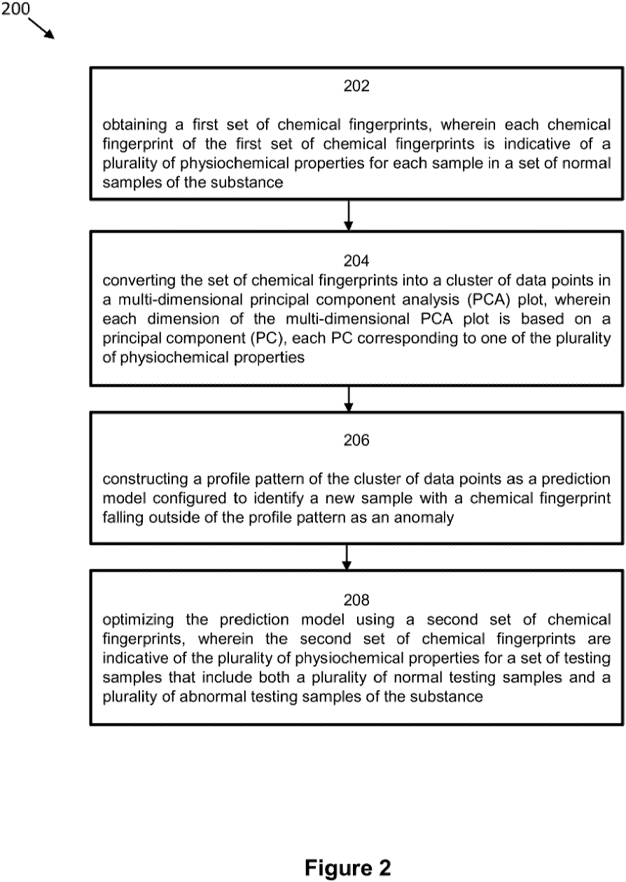

The device 100 at least includes one or more processor 102 and a memory 104. The at least one processor 102 and the memory 104 are interconnected. The memory 104 includes computer program code (not shown in FIG. 1) for execution by the at least one processor 102 to perform steps for anomaly detection as shown in FIG. 2 and described in the present application.

At step 202, the computer program code instructs the at least one processor 102 to obtain a first set of chemical fingerprints. Each chemical fingerprint of the first set of chemical fingerprints is indicative of a plurality of physiochemical properties for each sample in a set of normal samples of the substance.

In an embodiment, the substance is milk. In an example, the set of normal samples of the substance are 63473 normal raw milk samples sampled with MilkoScan FT120 (FOSS Analytical, Denmark) from 2017 to 2019 by Mengniu Dairy (Group) Co. Ltd. (Helin, Inner Mongolia, China). Chemical fingerprints of the normal raw milk samples are taken by Fourier transform infrared (FTIR) spectroscopy and stored in a database. Such a database can be established on internal memory or storage components of the device 100 or storage spaces external to the device 100.

Each chemical fingerprint is indicative of multivariate nature of each sample. For example, the chemical fingerprint can include two data formats: (1) spectral data of each sample in terms of absorbance vs. wavenumber in a range of 1000-3550 cm⁻¹, and (2) compositional data with a plurality of compositional features (interchangeably referred to as physiochemical properties) of each sample. In an embodiment, the plurality of physiochemical properties include eight (8) physiochemical properties such as fat, protein, solid non-fat, total solid, lactose, relative density, freezing point depression, and acidity of milk.

It is understandable to those skilled in the art that the compositional data can include different sets of physiochemical properties for different species of milk. The chemical fingerprint will accordingly reflect such physiochemical properties.

In addition, as the present method can be used for detection of anomaly in substance other than milk, it is appreciable to those skilled in the art that the compositional data can include various sets of physiochemical properties for various types of substance (either food items or non-food items). The chemical fingerprint will accordingly reflect such physiochemical properties.

In the present embodiment, the first set of chemical fingerprints include 63473 chemical fingerprints of the set of 63473 normal raw milk samples, each chemical fingerprint being indicative of the eight physiochemical properties and/or spectral data of each sample in the set of 63473 normal raw milk samples. At step 202, the first set of 63473 chemical fingerprints are obtained from the database.

At step 204, the computer program code instructs the at least one processor 102 to convert the first set of chemical fingerprints into a cluster of data points in a multi-dimensional principal component analysis (PCA) plot. Each dimension of the multi-dimensional PCA plot is based on a principal component (PC). In the present application, each PC corresponds to one of the plurality of physiochemical properties. In this sense, a PC can be interchangeably referred to as a physiochemical property in the following description. In the present application, this cluster of data points can be referred to as a first envelop for easy reference.

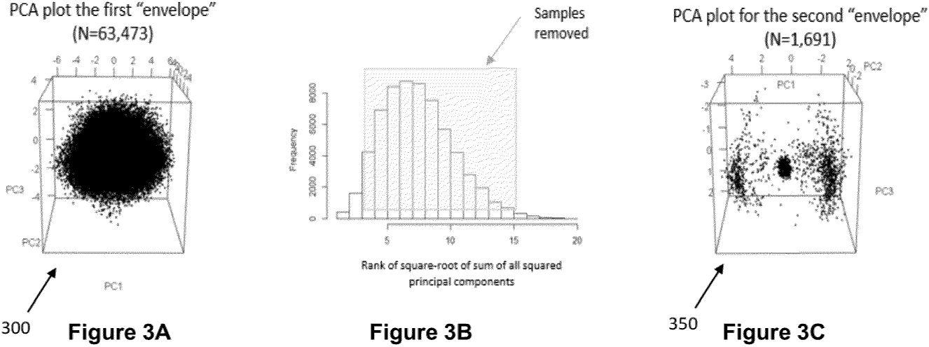

An embodiment of the converted PCA plot is depicted in FIG. 3A. As shown in FIG. 3A, a PCA plot 300 includes a cluster of data points for all the 63473 chemical fingerprints of the set of 63473 normal raw milk samples. However, this cluster is densely packed, which will require massive computation if all the data points in the cluster are included for constructing a prediction model. This technical challenge is advantageously solved by the following steps of the present application.

At step 206, the computer program code instructs the at least one processor 102 to construct a profile pattern of the cluster of data points as a prediction model. This prediction model can identify a new sample with a chemical fingerprint falling outside of the profile pattern as an anomaly.

For example, when the substance is milk, the anomaly includes one or more of potassium sulfate, potassium dichromate, citric acid, ammonium sulfate, melamine, urea, lactose, glucose, sucrose, maltodextrin and fructose, water, sodium citrate, and real-life scenarios such as milk with cow smell and milk being improperly stored for 36 hours. It is appreciable to those skilled in the art that when the anomaly varies, the variation is based on different substances. In an embodiment, if the prediction model receives a chemical fingerprint of a new milk sample that when converted into a new data point in the PCA plot falls outside the profile pattern in the PCA plot, the new milk sample will be identified as an anomaly by the prediction model. Likewise, if the prediction model receives a chemical fingerprint of a new milk sample that when converted into a new data point in the PCA plot falls on or inside the profile pattern in the PCA plot, the new milk sample will be identified as normal by the prediction model.

An embodiment of the step 206 of constructing the profile pattern is depicted in FIG. 3B. As shown, during the construction of the profile pattern, the computer program code instructs the at least one processor 102 to perform the following sub-steps:

First, at sub-step 206a, calculate a square foot of sum of all squared PCs for each of the data points in the cluster.

Thereafter, at sub-step 206b, rank the cluster of data points into a predetermined number of intervals based on their respective values of the calculated square foot of sum.

Thereafter, at sub-step 206c, obtain the profile pattern in the PCA plot by removing data points that fall in one or more ranks that have more than a predetermined number of data points from the cluster.

In an embodiment shown in FIG. 3B, the predetermined number of intervals is 20 and the predetermined number of data points is 1000. It is understandable to those skilled in the art that the predetermined numbers can vary and can be determined based on practical needs. In this embodiment, a total of 61,782 data points is removed from the PCA plot according to sub-steps 206a, 206b and 206c, and a profile pattern 350 is constructed by the remaining 1691 data points in the PCA plot. Such a profile pattern 350 shows “core” and “skin” of the cluster of data points, which maintains the structure of the cluster of data points. In the present application, the constructed profile pattern can be referred to as a second envelop for easy reference.

In the above described manner, the constructed profile pattern advantageously enables the present application to significantly reduce the required computation from 63473 data points to only 1691 data points while retaining the structure of the cluster of data points for all the 63473 chemical fingerprints. That is, it improves computational efficiency while maintaining accuracy of the prediction model.

At step 208, the computer program code instructs the at least one processor 102 to optimize the prediction model using a second set of chemical fingerprints of a set of testing samples that include both a plurality of normal testing samples and a plurality of abnormal testing samples.

In an embodiment, the set of testing samples includes 1087 testing samples which comprise 976 normal raw milk samples and 111 abnormal raw milk samples. Among the abnormal raw milk samples, the following chemicals of various concentrations are spiked in to mimic real-life adulterants (ADs): potassium sulfate, potassium dichromate, citric acid, ammonium sulfate, melamine, urea, lactose, glucose, sucrose, maltodextrin and fructose, water, sodium citrate, and real-life scenarios such as milk with cow smell and milk being improperly stored for 36 hours.

Table 1 shows the number of abnormal samples in the set of testing samples spiked with respective concentrations of adulterants.

TABLE 1

Number of abnormal samples with spiked adulterants added

| Adulterants (AD) added | Number of abnormal samples |
|---|---|
| Common chemicals | |
| Potassium dichromate* | 9 |
| Potassium sulfate* | 9 |
| Sodium citrate | 4 |
| Citric acid* | 9 |

| | |
|---|---|
| Nitrogen-based adulterants | |
| Ammonium sulfate* | 5 |
| Urea* | 5 |
| Melamine* | 10 |

| | |
|---|---|
| Carbohydrate-based adulterants | |
| Sucrose# | 5 |
| Glucose# | 5 |
| Lactose# | 5 |
| Fructose# | 5 |
| Maltodextrin# | 5 |
| Improperly stored for 36 hr | 23 |
| Water | 2 |
| Cow smell | 10 |
| Total | 111 |

*Adulterants added at concentrations 0.01, 0.02, 0.05, 0.1, 0.2 g per 100 g raw milk; #Adulterants added at concentrations 0.1, 0.2, 0.5, 1, 2 g per 100 g raw milk.

Similar to the first set of chemical fingerprints, each chemical fingerprint in the second set of chemical fingerprints is indicative of the multivariate nature of each normal or abnormal testing sample in the set of testing samples. For example, the chemical fingerprint can include two data formats: (1) spectral data of each sample in terms of absorbance vs. wavenumber in a range of 1000-3550 cm⁻¹, and (2) compositional data with a plurality of physiochemical properties of each normal or abnormal testing sample. In an embodiment, the plurality of physiochemical properties include eight (8) physiochemical properties such as fat, protein, solid non-fat, total solid, lactose, relative density, freezing point depression, and acidity of each normal raw milk sample or each abnormal raw milk sample.

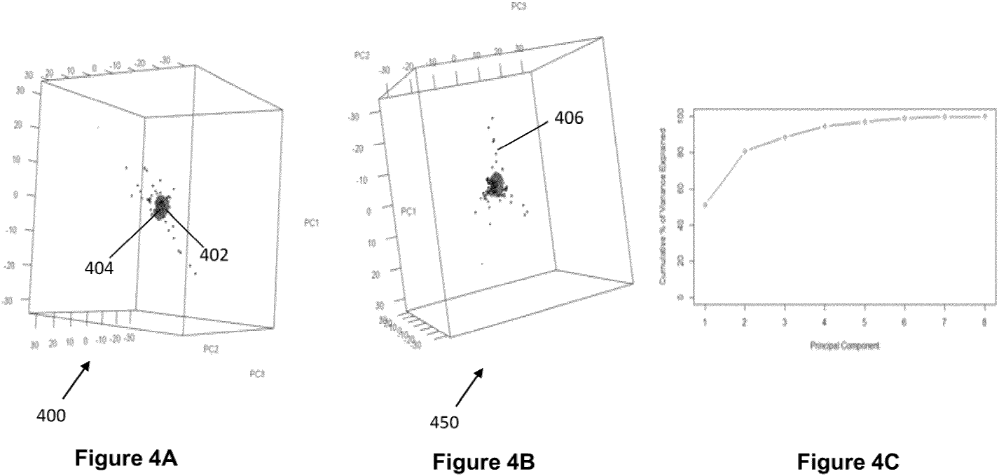

An embodiment of the step 208 of optimization of the prediction model are reflected in FIGS. 4A to 4C. As shown, during the optimization of the prediction model, the computer program code instructs the at least one processor 102 to perform the following sub-steps:

First, at sub-step 208a, for the set of testing samples, convert the second set of chemical fingerprints into new data points in the PCA plot. An embodiment of the new data points converted from the second set of chemical fingerprints is depicted in dots that are wrapped by an envelop shape 402 and dots found outside the envelop shape 402 in the PCA plot 400, 450 as shown in alternative angles of views in FIG. 4A and FIG. 4B. In the PCT plot 400, 450, the profile pattern obtained at step 206 is depicted in dots that form the envelope shape 402.

Thereafter, at sub-step 208b, for each sample in the set of testing samples, calculate a squared Mahalanobis distance (MD) score between the sample to a centroid in the profile pattern in the PCA plot and determine a threshold squared MD score to distinguish whether the sample falls inside or outside of the profile pattern.

In an embodiment, the squared MD score can be calculated by a MD score calculation script on R or RStudio as follows, taking top X number of Principle Components (PCs) in consideration. It is understood that names in italic fonts can be replaced by own names based on practical needs:

name1<-prcomp(matrixdataframe,scale=TRUE)
name2<- as.data.frame(name1$x[, 1:X])
MD <- mahalanobis(name2, center = colMeans(name2), cov = cov(name2))
name2$MD <- round(MD, 3)

As shown in the embodiment of FIG. 4A, samples (depicted in dots that are wrapped by the envelop shape 402) wrapped inside the profile pattern (depicted in dots that form the envelope shape 402) are normal testing samples from the set of testing samples, while FIG. 4B shows that samples ( for example, depicted in dots such as dot 406) found outside the profile pattern are abnormal testing samples from the set of testing samples.

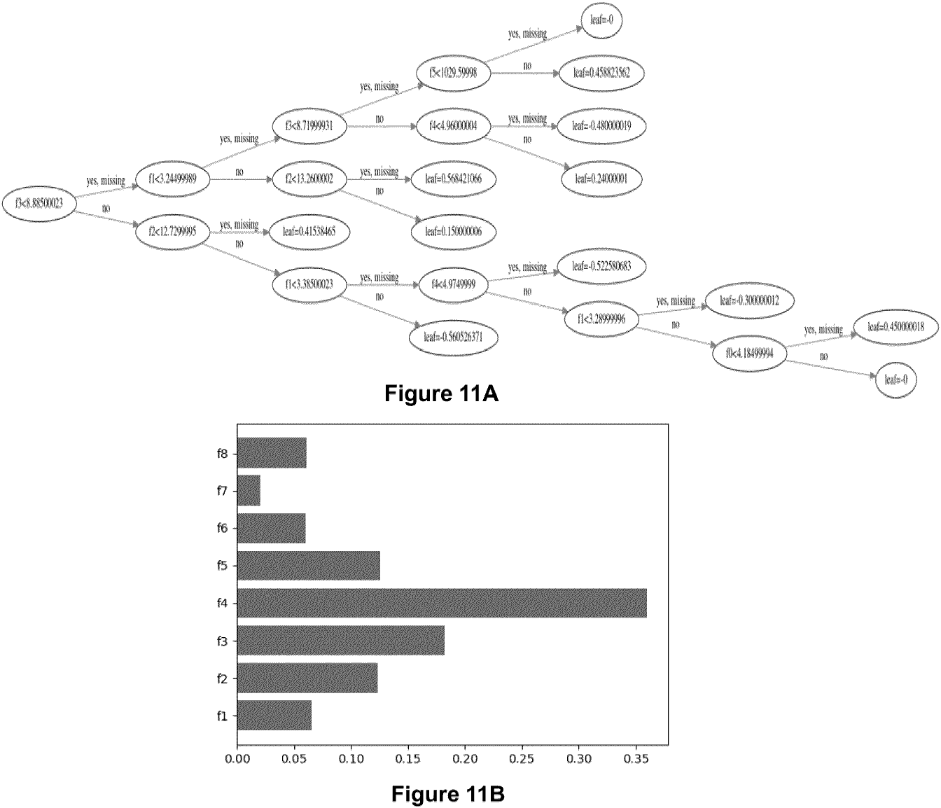

FIG. 4C shows a scree plot of cumulative % variances for the profile pattern with various numbers (from 1 to 8 in this example) of principal components (PCs). In FIG. 4C, it is shown that four PCs are accountable for 94.9% of the variance of the profile pattern. Therefore, the present application can advantageously improve computational efficiency by using only the four most important PCs for the squared MD score calculations in sub-step 208b. Details of the four most important PCs are described with respect to FIGS. 11A and 11B.

In addition, as shown in Table 4 below, compared to the cluster of data points for the first set of chemical fingerprints (i.e. the first envelope), the profile pattern (i.e. the second envelope) yields better accuracy (95.22% vs. 89.97%), specificity (99.45% vs. 91.68%), precision (96.21 % vs. 64.98%) and CSI (70.95% vs. 56.40%), with a comparable BA (86.22% vs. 86.36%), albeit with a lower sensitivity (72.99% vs. 81.03%). It shows that the prediction model using squared MD score calculation based on the 4 most important PCs in sub-step 208b can achieve an accurate prediction result by effectively distinguish between normal and abnormal samples in an industrial dairy setting, which has a superior performance to national standards.

In an embodiment, in sub-step 208b, the computer program code instructs the at least one processor 102 to perform the following sub-steps to determine the threshold squared MD score.

First, in sub-step 208b1, rank squared MD scores for the set of testing samples.

Thereafter, in sub-step 208b2, iterate a range of squared MD scores as threshold values in a sensitivity calculation.

Thereafter, in sub-step 208b3, determine a squared MD score having a highest sensitivity value and an overall accuracy over 95% in the iterated range of squared MD scores as the threshold squared MD score.

In some embodiments, the sensitivity value is calculated based on the following equation:

$$\begin{matrix}\text{Sensitivity value =} \\ \frac{\text{Number of samples correctly identified as anomaly}}{\text{Number of samples with squared MD score greater than threshold value}} \times 100 \% .\end{matrix}$$

In some embodiments, the overall accuracy is calculated based on the following equation:

$$\begin{matrix}\text{Overall accuracy} = \\ \frac{\text{Number of samples correctly identified as true normal samples and anomaly}}{\text{Number of all samples in the set of testing samples}} \times 100 \% .\end{matrix}$$

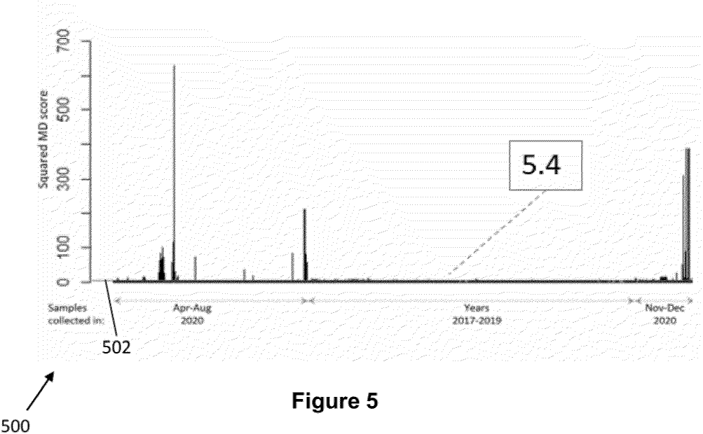

FIG. 5 shows a diagram 500 depicting a distribution of squared Mahalanobis distance (MD) scores for each sample in the set of testing samples set, according to an embodiment. In the embodiment, the line 502 represents a threshold squared MD score of 5.4 to distinguish whether a sample falls inside (normal) or outside (abnormal) of the profile pattern.

Table 2 shows overall accuracy, sensitivity and specificity, precision (or positive predictive value), negative predictive value (NPV), critical success index (CSI) and the balanced accuracy (BA) when a threshold MD score of 5.4 is used in the sub-step 208b to distinguish whether each testing sample in the set of testing samples falls inside (normal) or outside (abnormal) of the profile pattern. In the embodiment, the threshold squared MD score 5.4 provides an overall accuracy, sensitivity and specificity of 95.86%, 72.99% and 97.39% respectively. In addition, the precision (or positive predictive value) is 65.13%, the negative predictive value (NPV) is 98.18%, the critical success index (CSI) is 52.48% and the balanced accuracy (BA) is 85.19%.

TABLE 2

| | | |
|---|---|---|
| Correct | True Normal Prediction | 2536 |
| | True Anomaly (AD) | 127 |
| Mis-Prediction | False Normal | 47 |
| | False Anomaly (AD) | 68 |
| Accuracy | Prediction metrics | 95.86% |
| | Sensitivity | 72.99% |
| | Specificity | 97.39% |
| | Precision | 65.13% |
| | Negative Predictive Value (NPV) | 98.18% |
| | Critical Success Index (CSI) | 52.48% |
| | Balanced Accuracy (BA) | 85.19% |

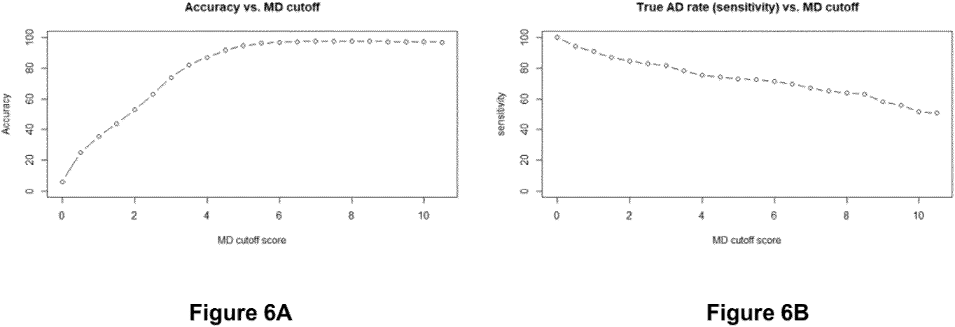

FIG. 6A shows a diagram depicting an overall accuracy when iterating a range of squared MD scores for determining a threshold squared MD score. FIG. 6B shows a diagram depicting a sensitivity value when iterating a range of squared MD scores for determining a threshold squared MD score. Sample grouping shows that a threshold squared MD score of 5.4 is very effective in identifying normal raw milk samples, with a correct prediction rate of 99.5%. This threshold squared MD score is also effective at detecting abnormal samples, giving a correct prediction rate of 94.6%, as shown in FIGS. 6A and 6B.

Table 3 shows detection limits for each anomaly (adulterant) in the set of testing samples when a threshold MD score of 5.4 is used in the sub-step 208b. Comparison of the squared MD scores between normal samples and abnormal samples using Welch Two Sample t-test (2-sided) produces a p-value of 6.523 × 10⁻⁸, and all squared MD scores of normal samples in the set of testing samples lies within the profile pattern. In addition, squared MD scores can even detect abnormal samples that had passed the national standard: t-test comparing between “spiked abnormal samples within national standards” (N=44) to normal samples gives a p-value of 0.01359. Among the spiked abnormal samples, raw milk diluted with water (N=2) and raw milk of poor quality (with “cow smell”, N=10) are readily detected with a correct prediction rate reaching 100%. The majority of the raw milk deteriorated due to improper storage conditions (N=23) and raw milk spiked with various buffering reagents or inorganic chemicals (potassium sulfate, potassium dichromate, citric acid, sodium citrate) can also be detected, resulting in a correct prediction rate of 87.0% and 71.0% respectively. Glucose, maltodextrin and fructose are detectable at 2% and sucrose could only be detected at 1%. While as low as 0.02% (w/w) melamine could be detected, ammonium sulfate and urea are detectable at 0.1% and 0.2% respectively. Nitrogen-rich (ammonium sulfate, melamine, urea) or carbohydrate-based (lactose, glucose, Sucrose, maltodextrin, fructose) adulterants are less readily detected. Lactose is undetectable even at 2% concentration.

TABLE 3

| Adulterants added | Number of raw milk samples | Cutoff concentration (g/100 g of raw milk) |
|---|---|---|
| Common chemicals: | | |
| Potassium dichromate* | 9 | 0.2 |
| Potassium sulfate* | 9 | 0.1 |
| Sodium citrate | 4 | 0.01 |
| Citric acid* | 9 | 0.05 |
| Nitrogen-based adulterants | | |
| Ammonium sulfate* | 5 | 0.1 |
| Urea* | 5 | 0.2 |
| Melamine* | 10 | 0.02 |
| Carbohydrate-based adulterants: | | |
| Sucrose# | 5 | 1 |
| Glucose# | 5 | 2 |
| Lactose# | 5 | Undetected |
| Fructose# | 5 | 2 |
| Maltodextrin# | 5 | 2 |
| Improperly stored for 36 hr | 23 | Correct prediction rate of 87% |
| Water | 2 | Correct prediction rate of 100% |
| Cow smell | 10 | Correct prediction rate of 100% |

To compare the performance of the second “envelope” with the first “envelope”, a cutoff squared MD score of 8.6 is obtained by applying the sub-step 208b to a centroid in the cluster of data points (the first envelope) instead of a centroid in the pattern profile (the second envelope). Although the inclusion of more samples by the first envelope resulted in higher overall accuracy and sensitivity, precision and CSI are considerably lowered, and sucrose becomes undetectable. Therefore, the prediction model using the pattern profile (the second envelope) according to the present application outperforms a prediction model using the cluster of data points of the first set of chemical fingerprints (the first envelope) in terms of accuracy, specificity, precision and critical success index (CSI), as shown in Table 4 below.

TABLE 4

Comparison of the prediction performance between first and second “envelopes”

| Prediction Model Approach | | Profile Pattern of the cluster of data points (I.E. Second Envelop) | Cluster of data points of the first set of chemical fingerprints (i.e. First Envelop) |
|---|---|---|---|
| Threshold squared MD score (i.e. squared MD cutoff score) | | 5.4 | 8.6 |
| Correct Prediction | True Normal | 908 | 837 |
| | True Anomaly (AD) | 127 | 141 |
| Mis-Prediction | False Normal | 47 | 33 |
| | False Anomaly (AD) | 5 | 76 |
| Prediction Metrics | Accuracy | 95.22% | 89.97% |
| | Sensitivity | 72.99% | 81.03% |
| | Specificity | 99.45% | 91.68% |
| | Precision | 96.21% | 64.98% |
| | Negative Predictive Value (NPV) | 95.08% | 96.21% |
| | Critical Success Index (CSI) | 70.95% | 56.40% |
| | Balanced Accuracy (BA) | 86.22% | 86.36% |

In some embodiments, to facilitate the calculation for the overall accuracy calculation, the computer program code can further instruct the at least one processor 102 to train the prediction model to define true normal samples based on hierarchical cluster learning of spectral data of the set of testing samples.

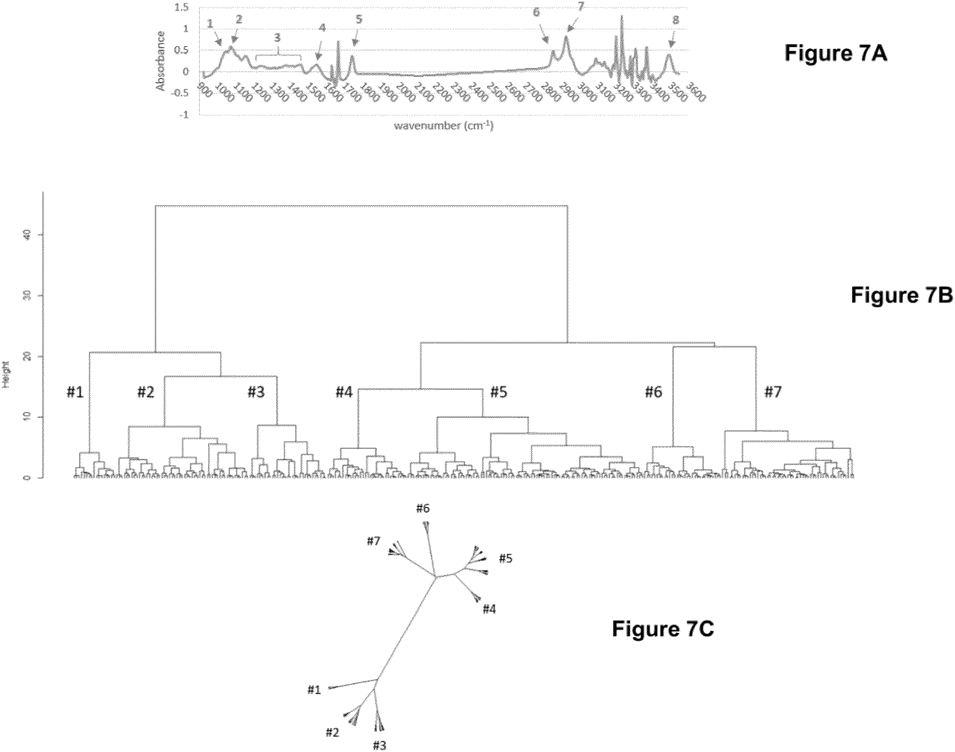

FIG. 7A is a diagram showing spectral data of raw milk retrieved from Fourier transform infrared (FTIR) spectroscopy. As described above, the spectral data can be comprised in the chemical fingerprint of each sample. In this diagram, the spectral data is plotted in terms of absorbance vs. wavenumber. In FIG. 7A, arrowed numbers 1 to 8 refer to eight (8) spectrum regions where absorbance values are extracted for hierarchical cluster learning to define true normal samples.

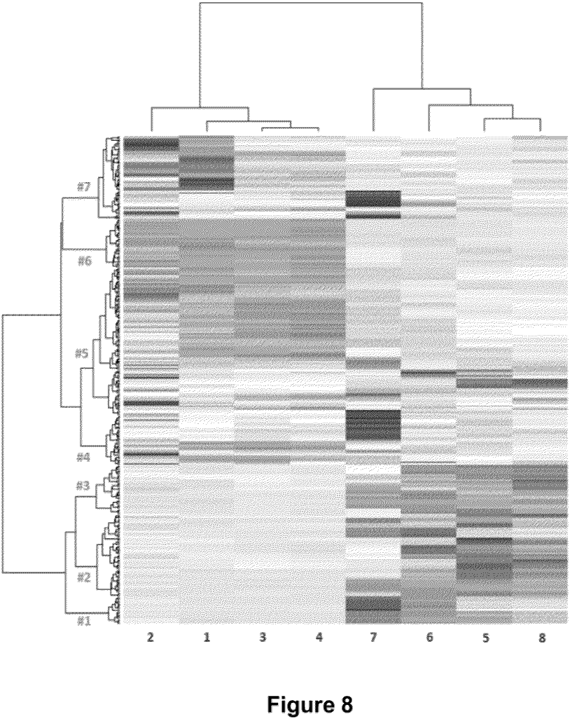

In an embodiment, to test the effectiveness of classifying stringent abnormal samples, unsupervised hierarchical clustering is performed in 40 abnormal samples with low concentrations of AD that had passed national standards and 68 normal samples. As shown in FIG. 7A, the absorbance values for seven peaks within the spectrum regions 1000-1100, 1500-1600, 1730-1800, 2840-2940, 3450-3550 cm⁻¹ and the averaged absorbance value for 1250-1450 cm⁻¹ are extracted from the spectral data for each sample. Unsupervised hierarchical clustering with Euclidean distance and Ward’s linkage method are performed. Clustering is visualized by dendrograms and heat-maps as shown in FIGS. 7B-7C and FIG. 8.

FIG. 7B is a dendrogram showing samples clusters within main branches in the hierarchical cluster learning. FIG. 7B shows that carbohydrate-based adulterants cluster in branches #1 & #6, nitrogen-rich adulterants cluster in branch #7, buffering reagents or inorganic chemicals cluster in branch #4, while normal samples cluster in branches #2, #3, #5.

FIG. 7C is an un-rooted tree showing that two major clusters are observed by the hierarchical cluster learning. Among the two major clusters, one cluster includes branches #1-3 and the other cluster includes branches #4-7.

FIG. 8 shows a heat-map visualizing hierarchical cluster learning of 113 abnormal samples (i.e. anomalies) classified as within national standards and 198 true normal samples. In FIG. 8, numbers on the left of the figure indicate branch # of dendrogram for samples, whereas numbers on the bottom of the figure indicate regions where absorbance values are extracted from. FIG. 8 shows that sub-classes exist among normal raw milk samples and carbohydrate-based abnormal samples in clusters #2, #3 and #5, and branches #1 and #6 respectively. On the other hand, nitrogen-rich adulterants and buffering reagents exert a stronger effect on the FTIR spectra that only one major branch is observed.

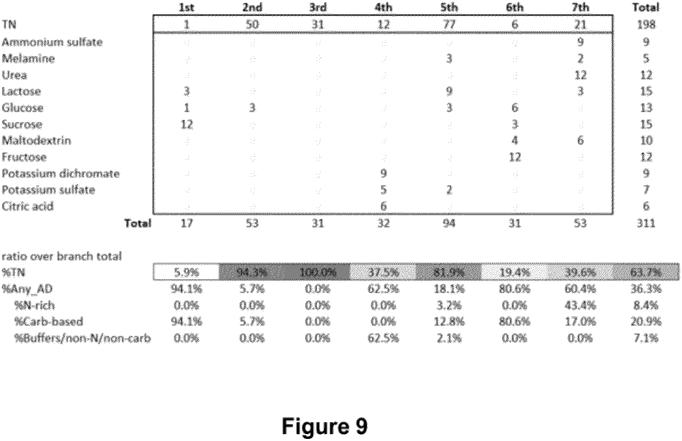

FIG. 9 shows a distribution of different anomaly compositions among the 7 branches (TN: True normal, AD: adulterant, N: Nitrogen, Carb: carbohydrate). FIG. 9 further shows that the first cluster contains mostly normal samples (true normal% = 81.2%; total = 101, true normal = 82, carbohydrate-based AD = 19), while the second cluster contains more diverse types of abnormal samples (True normal% = 55.2%; Total = 210, True normal = 116, nitrogen-rich AD = 26, carbohydrate-based AD = 46, buffering reagents = 22).

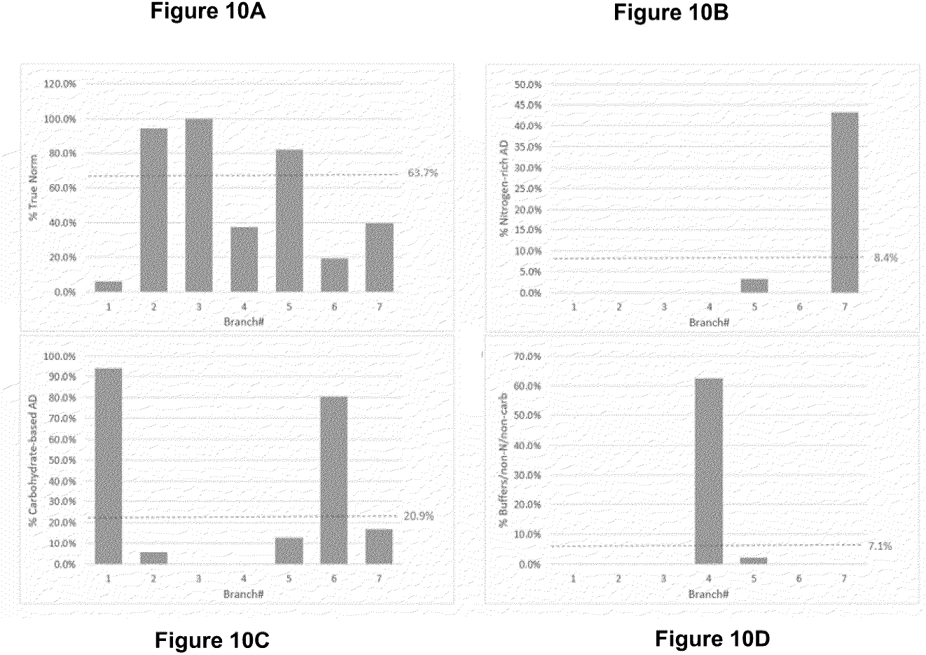

FIG. 10A is a diagram showing ratio of true normal samples within respective branches. FIG. 10B is a diagram showing ratio of nitrogen-rich abnormal samples within respective branches. FIG. 10C is a diagram showing ratio of carbohydrate-based abnormal samples within respective branches. FIG. 10C is a diagram showing ratio of buffering reagents as abnormal samples within respective branches. In FIGS. 10A to 10D, dotted lines and the accompanying % indicate overall % of true normal (198/311 = 63.7%) in FIG. 10A, nitrogen-rich abnormal samples (26/311 = 8.4%) in FIG. 10B, carbohydrate-based abnormal samples (65/311 = 20.9%) in FIG. 10C, and buffering reagents (22/311 = 7.1%) in FIG. 10D.

In some embodiments, to optimize the overall accuracy, the computer program code can further instruct the at least one processor 102 to train the prediction model based on Extratree or XGBoost learning of the plurality of physiochemical properties of the set of testing samples. It is learnt by the prediction model that certain physiochemical properties are more indicative of the profile pattern than other physiochemical properties.

FIG. 11A shows a boosting tree of a XGBoost model learned by the present prediction model, according to an embodiment.

FIG. 11B is a diagram showing relative importance of each compositional feature (that is, physiochemical property) f1 - f9 according to the learnt XGBoost model. f1 - f9 stands for fat, protein, total solid, non-fat solid, lactose, relative density, freezing point, acidity respectively.

Both FIGS. 11A and 11B show that the non-fat solid is the most indicative factor to determine whether the raw milk samples are treated; whereas protein and total solid lactose are the most indicative factors to detect potentially adulterated or contaminated samples as anomaly.

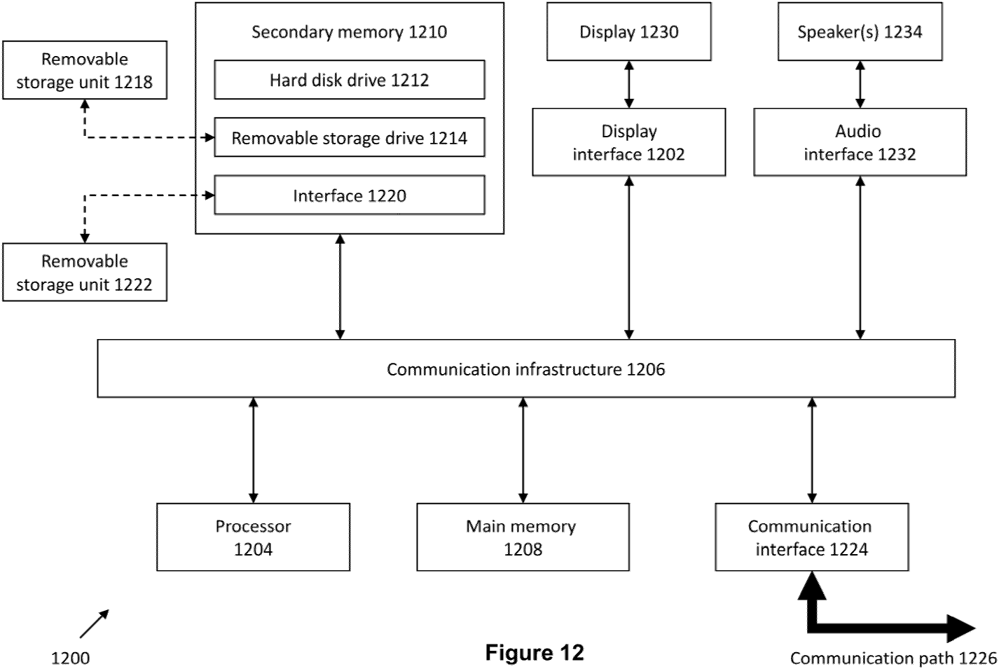

FIG. 12 shows a block diagram of a computer system 1200 suitable for use as a device 100 for anomaly detection of a substance as described herein.

The following description of the computer system / computing device 1200 is provided by way of example only and is not intended to be limiting.

As shown in FIG. 12, the example computing device 1200 includes a processor 1204 for executing software routines. Although a single processor is shown for the sake of clarity, the computing device 1200 may also include a multi-processor system. The processor 1204 is connected to a communication infrastructure 1206 for communication with other components of the computing device 1200. The communication infrastructure 1206 may include, for example, a communications bus, cross-bar, or network.

The computing device 1200 further includes a main memory 1208, such as a random access memory (RAM), and a secondary memory 1210. The secondary memory 1210 may include, for example, a hard disk drive 1212 and/or a removable storage drive 1214, which may include a magnetic tape drive, an optical disk drive, or the like. The removable storage drive 1214 reads from and/or writes to a removable storage unit 1218 in a well-known manner. The removable storage unit 1218 may include a magnetic tape, optical disk, or the like, which is read by and written to by removable storage drive 1214. As will be appreciated by persons skilled in the relevant art(s), the removable storage unit 1218 includes a computer readable storage medium having stored therein computer executable program code instructions and/or data.

In an alternative implementation, the secondary memory 1210 may additionally or alternatively include other similar means for allowing computer programs or other instructions to be loaded into the computing device 1200. Such means can include, for example, a removable storage unit 1222 and an interface 1220. Examples of a removable storage unit 1222 and interface 1220 include a removable memory chip (such as an EPROM or PROM) and associated socket, and other removable storage units 1222 and interfaces 1220 which allow software and data to be transferred from the removable storage unit 1222 to the computer system 1200.

The computing device 1200 also includes at least one communication interface 1224. The communication interface 1224 allows software and data to be transferred between computing device 1200 and external devices via a communication path 1226. In various embodiments, the communication interface 1224 permits data to be transferred between the computing device 1200 and a data communication network, such as a public data or private data communication network. The communication interface 1224 may be used to exchange data between different computing devices 1200 which such computing devices 1200 form part an interconnected computer network. Examples of a communication interface 1224 can include a modem, a network interface (such as an Ethernet card), a communication port, an antenna with associated circuitry and the like. The communication interface 1224 may be wired or may be wireless. Software and data transferred via the communication interface 1224 are in the form of signals which can be electronic, electromagnetic, optical or other signals capable of being received by communication interface 1224. These signals are provided to the communication interface via the communication path 1226.

Optionally, the computing device 1200 further includes a display interface 1202 which performs operations for rendering images to an associated display 1230 and an audio interface 1232 for performing operations for playing audio content via associated speaker(s) 1234.

As used herein, the term “computer program product” may refer, in part, to removable storage unit 1218, removable storage unit 1222, a hard disk installed in hard disk drive 1212, or a carrier wave carrying software over communication path 1226 (wireless link or cable) to communication interface 1224. Computer readable storage media refers to any non-transitory tangible storage medium that provides recorded instructions and/or data to the computing device 1200 for execution and/or processing. Examples of such storage media include floppy disks, magnetic tape, CD-ROM, DVD, Blu-ray™ Disc, a hard disk drive, a ROM or integrated circuit, USB memory, a magneto-optical disk, or a computer readable card such as a PCMCIA card and the like, whether or not such devices are internal or external of the computing device 1200. Examples of transitory or non-tangible computer readable transmission media that may also participate in the provision of software, application programs, instructions and/or data to the computing device 1200 include radio or infra-red transmission channels as well as a network connection to another computer or networked device, and the Internet or Intranets including e-mail transmissions and information recorded on Websites and the like.

The computer programs (also called computer program code) are stored in main memory 1208 and/or secondary memory 1210. Computer programs can also be received via the communication interface 1224. Such computer programs, when executed, enable the computing device 1200 to perform one or more features of embodiments discussed herein. In various embodiments, the computer programs, when executed, enable the processor 1204 to perform features of the above-described embodiments. Accordingly, such computer programs represent controllers of the computer system 1200.

Software may be stored in a computer program product and loaded into the computing device 1200 using the removable storage drive 1214, the hard disk drive 1212, or the interface 1220. Alternatively, the computer program product may be downloaded to the computer system 1200 over the communications path 1226. The software, when executed by the processor 1204, causes the computing device 1200 to perform functions of embodiments described herein.

It is to be understood that the embodiment of FIG. 12 is presented merely by way of example. Therefore, in some embodiments one or more features of the computing device 1200 may be omitted. Also, in some embodiments, one or more features of the computing device 1200 may be combined together. Additionally, in some embodiments, one or more features of the computing device 1200 may be split into one or more component parts.

The techniques described in this specification produce one or more technical effects. As mentioned above, embodiments of the present application provide approaches for non-targeted detection of anomaly in a substance. The approaches utilise machine learning methods to learn chemical fingerprints of a substance so as to construct and optimise a prediction model to identify an anomaly, either previously encountered or un-encountered. In the present application, an anomaly is interchangeably referred to as an abnormal sample or an adulterant (AD).

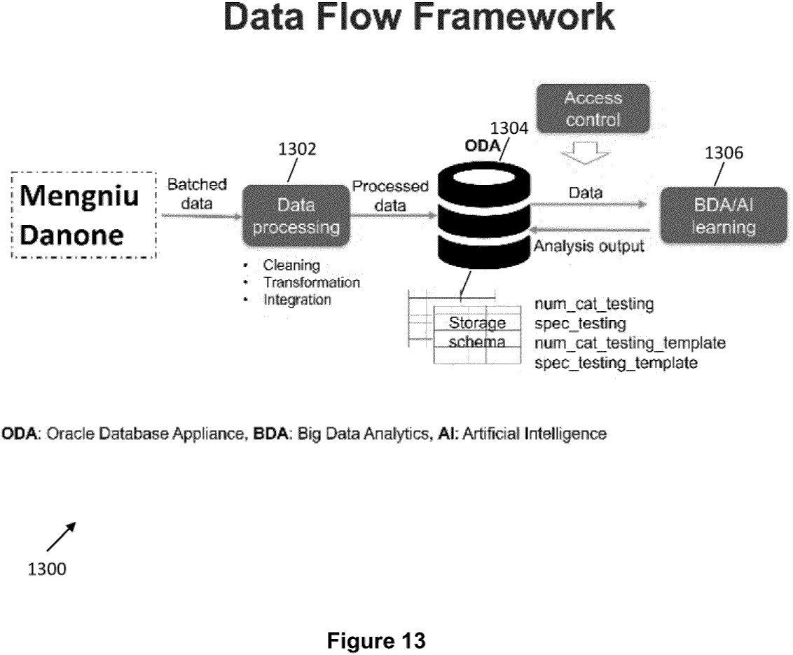

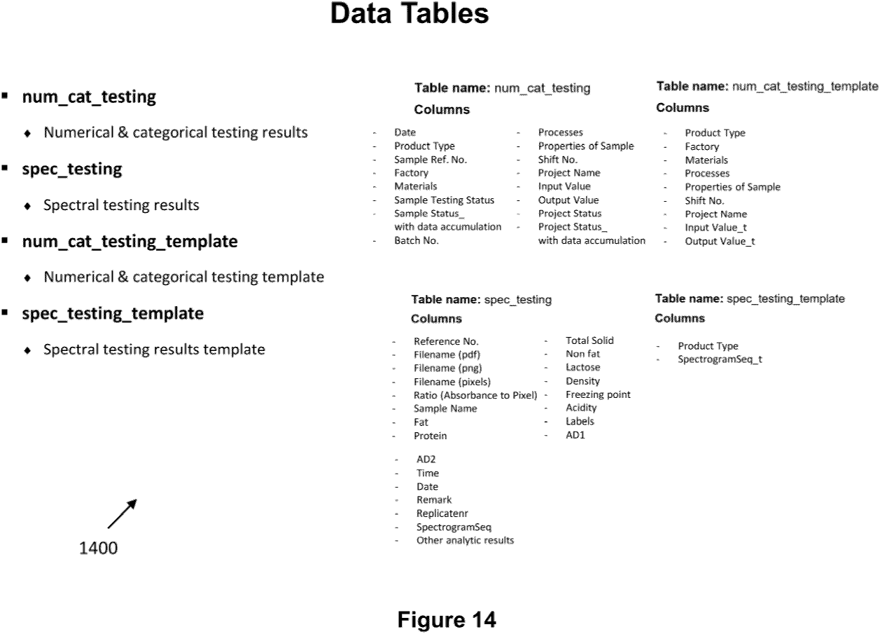

Furthermore, as appreciated by those skilled in the art, continuous updates with chemical fingerprints for new milk samples can be made to train the prediction model. The updates may include as many factors as possible, which include key chemometrics and all known characteristics of cattle such as geographic-seasonal-logistic variations, cow breeds, feeds, age, etc. and information on erroneous addition of specific ingredients such as non-dairy protein, illegal preservatives, high level of legal preservatives, antibiotics, pesticide. Chemical fingerprints should be extended beyond FTIR to also cover mass spectrometry, MMR, infrared spectroscopy, liquid chromatography, gas chromatography etc. Chemical fingerprints can be augmented to also include biological fingerprints such as data from next-generation sequencing (NGS) to provide valuable fraudulent information such as milk of different species, vegetable and animal fats; fraudulent claim on breed and geographical upbringing; and contamination of pathogenic microbes. The chemical fingerprints and biological fingerprints can be in various heterogeneous reporting formats comprising numerical values, data points, graphs, images, digital representations, etc. These chemical fingerprints and biological fingerprints can be converted into a standardized format in the form of schemas to facilitate data storage in the database. An embodiment of the schemas is shown in FIGS. 13 and 14. In FIG. 13, the chemical fingerprints and biological fingerprints are referred to as “data” in an exemplary data flow framework 1300. In FIG. 14, exemplary data tables are shown with columns of data captured in the respective schemas num_cat_testing, spec_testing, num_cat-testing_template, and spec_testing_template as depicted in FIG. 13.

These data can be processed in a data processing modular 1302 and converted into a standardized format for being stored in a database 1304. The standardized format includes schemas about numerical and categorical testing results (num_cat_testing), spectral testing results (spec_testing), numerical and categorical testing templates (num_cat_testing_template), and spectral testing results templates (spec_testing_template). It is appreciable to those skilled in the art that the standardized format may include other schemas about other physiochemical properties of samples. The stored data in the standardized format can be provided to a machine learning module 1306 to train a prediction model for anomaly detection of a substance based on the embodiments as describe in the preceding paragraphs.

Upon continuous machine learning, the predictive power of the prediction model will be further enhanced, such that the approaches provided in the present application can be applied as standard screening for a wide range of food commodities.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1. A method for anomaly detection of a substance, the method comprising:
obtaining a first set of chemical fingerprints, wherein each chemical fingerprint of the first set of chemical fingerprints is indicative of a plurality of physiochemical properties for each sample in a set of normal samples of the substance;
converting the first set of chemical fingerprints into a cluster of data points in a multi-dimensional principal component analysis (PCA) plot, wherein each dimension of the multi-dimensional PCA plot is based on a principal component (PC), each PC corresponding to one of the plurality of physiochemical properties;
constructing a profile pattern of the cluster of data points as a prediction model configured to identify a new sample with a chemical fingerprint falling outside of the profile pattern as an anomaly; and
optimizing the prediction model using a second set of chemical fingerprints, wherein the second set of chemical fingerprints are indicative of the plurality of physiochemical properties for a set of testing samples that include both a plurality of normal testing samples and a plurality of abnormal testing samples of the substance.

2. The method according to claim 1, wherein the constructing of the profile pattern comprises:
for each of the data points in the cluster, calculating a square foot of sum of all squared PCs;
ranking the cluster of data points into a predetermined number of intervals based on their respective values of the calculated square foot of sum; and
obtaining the profile pattern in the PCA plot by removing data points that fall in one or more ranks that have more than a predetermined number of data points from the cluster.

3. The method according to claim 2, wherein the predetermined number of intervals is 20 and the predetermined number of data points is 1000.

4. The method according to claim 1, wherein the optimizing of the prediction model comprises:
for the set of testing samples, converting the second set of chemical fingerprints into new data points in the PCA plot; and
for each sample in the set of testing samples, calculating a squared Mahalanobis distance (MD) score between the sample to a centroid in the profile pattern in the PCA plot and determining a threshold squared MD score to distinguish whether the sample falls inside or outside of the profile pattern.

5. The method according to claim 4, wherein the determining of the threshold squared MD score comprises:
ranking squared MD scores for the set of testing samples;
iterating a range of squared MD scores as threshold values in a sensitivity calculation; and
determining a squared MD score having a highest sensitivity value and an overall accuracy over 95% in the iterated range of squared MD scores as the threshold squared MD score.

6. The method according to claim 5, wherein the sensitivity value is calculated based on the following equation:
$$\begin{matrix}\text{Sensitivity value} = \\ \frac{\text{Number of samples correctly identified as anomaly}}{\begin{matrix}\text{Number of samples with squared MD score} \\ \text{greater than threshold value}\end{matrix}} \times 100 \% .\end{matrix}$$
.

7. The method according to claim 5, wherein the overall accuracy is calculated based on the following equation:
$$\begin{matrix}\text{Overall accuracy} = \\ \frac{\begin{matrix}\text{Number of samples correctly identified as} \\ \text{true normal samples and anomaly}\end{matrix}}{\text{Number of all samples in the set of testing samples}} \times 100 \% .\end{matrix}$$
.

8. The method according to claim 5, further comprising:
training the prediction model to define true normal samples based on hierarchical cluster learning of spectral data of the set of testing samples.

9. The method according to claim 5, further comprising:
training the prediction model to optimize the overall accuracy based on Extratree or XGBoost learning of the plurality of physiochemical properties of the set of testing samples.

10. The method according to claim 1, wherein the substance is milk.

11. A device for anomaly detection of a substance, the device comprising:
at least one processor; and
a memory including computer program code for execution by the at least one processor, wherein the computer program code instructs the at least one processor to:
obtain a first set of chemical fingerprints, wherein each chemical fingerprint of the first set of chemical fingerprints is indicative of a plurality of physiochemical properties for each sample in a set of normal samples of the substance;
convert the first set of chemical fingerprints into a cluster of data points in a multi-dimensional principal component analysis (PCA) plot, wherein each dimension of the multi-dimensional PCA plot is based on a principal component (PC), each PC corresponding to one of the plurality of physiochemical properties;
construct a profile pattern of the cluster of data points as a prediction model configured to identify a new sample with a chemical fingerprint falling outside of the profile pattern as an anomaly; and
optimize the prediction model using a second set of chemical fingerprints, wherein the second set of chemical fingerprints are indicative of the plurality of physiochemical properties for a set of testing samples that include both a plurality of normal testing samples and a plurality of abnormal testing samples of the substance.

12. The device according to claim 11, wherein during the constructing of the profile pattern, the computer program code further instructs the at least one processor to:
for each of the data points in the cluster, calculate a square foot of sum of all squared PCs;
rank the cluster of data points into a predetermined number of intervals based on their respective values of the calculated square foot of sum; and
obtain the profile pattern in the PCA plot by removing data points that fall in one or more ranks that have more than a predetermined number of data points from the cluster.

13. The device according to claim 12, wherein the predetermined number of intervals is 20 and the predetermined number of data points is 1000.

14. The device according to claim 11, wherein during the optimizing of the prediction model, the computer program code further instructs the at least one processor to:
for the set of testing samples, convert the second set of chemical fingerprints into new data points in the PCA plot; and
for each sample in the set of testing samples, calculate a squared Mahalanobis distance (MD) score between the sample to a centroid in the profile pattern in the PCA plot and determine a threshold squared MD score to distinguish whether the sample falls inside or outside of the profile pattern.

15. The device according to claim 14, wherein during the determining of the threshold squared MD score, the computer program code further instructs the at least one processor to:
rank squared MD scores for the set of testing samples;
iterate a range of squared MD scores as threshold values in a sensitivity calculation; and
determine a squared MD score having a highest sensitivity value and an overall accuracy over 95% in the iterated range of squared MD scores as the threshold squared MD score.

16. The device according to claim 15, wherein the sensitivity value is calculated based on the following equation:
$$\begin{matrix}\text{Sensitivity value} = \\ \frac{\text{Number of samples correctly identified as anomaly}}{\begin{matrix}\text{Number of samples with squared MD score} \\ \text{greater than threshold value}\end{matrix}} \times 100 \% .\end{matrix}$$
.

17. The device according to claim 15, wherein the overall accuracy is calculated based on the following equation:
$$\begin{matrix}\text{Overall accuracy} = \\ \frac{\begin{matrix}\text{Number of samples correctly identified as} \\ \text{true normal samples and anomaly}\end{matrix}}{\text{Number of all samples in the set of testing samples}} \times 100 \% .\end{matrix}$$
.

18. The device according to claim 15, wherein the computer program code further instructs the at least one processor to:
train the prediction model to define true normal samples based on hierarchical cluster learning of spectral data of the set of testing samples.

19. The device according to claim 11, wherein the substance is milk.

20. A non-transitory computer readable storage medium having instructions encoded thereon that, when executed by a processor, cause the processor to perform one or more steps in a method for anomaly detection of a substance according to claim 1.
